SEQUENCE LISTING (1 sequence)

SEQ ID NO: 1 - DNA, 422 bp, synthetic construct
  source 1..422: mol_type = other DNA
atggatgtgttcatgaaaggactttcaaaggccaaggagggagttgtggctgctgctgagaaaaccaagcagggtgtggcagaggcagctggaaagacaaaagagggagtcctctatgtaggttccaaaactaaggaaggagtggttcatggagtgacaacagtggctgagacgaccaaagagcaagtgacaaatgttggaggagcagtggtgactggtgtgacagcagtcgctcagaagacagtggagggagctgggaatatagctgctgccactggctttgtcaagaaggaccagatgggcaagggtgaggaggggtacccacaggaaggaatcctggaagacatgcctgtggatcctggcagtgaggcttatgaaatgccttcagaggaaggctaccaagactatgagcctgaagccta

(19) United States
(12) Patent Application Publication
(10) Pub. No.: US 2024/0402192 A1
(43) Pub. Date: Dec. 5, 2024

(54) KIT FOR QUIESCENT DETECTING ALPHA-SYNUCLEIN AGGREGATES

(71) Applicant: Pingyi XU, Guangzhou (CN)

(72) Inventors: Hengxu MAO, Guangzhou (CN); Pingyi XU, Guangzhou (CN); Yaoyun KUANG, Guangzhou (CN); Xinling YANG, Urumqi (CN); Lin LU, Guangzhou (CN); Wenyuan GUO, Guangzhou (CN); Hongyan LI, Urumqi (CN); Yuxuan YONG, Urumqi (CN); Wenjun WU, Zhongshan (CN); Xiaoyun HUANG, Dongguan (CN)

(21) Appl. No.: 18/451,639

(22) Filed: Aug. 17, 2023

(30) Foreign Application Priority Data
Jun. 1, 2023 (CN) 202310648092.3

(51) Int. Cl. G01N 33/68; G01N 1/28; G01N 1/38; G01N 33/52

(52) U.S. Cl. CPC G01N 33/6896; G01N 1/286; G01N 1/38; G01N 33/52; G01N 2333/435; G01N 2800/2828

(57) ABSTRACT

A kit for quiescent detecting alpha-synuclein aggregates is provided by the present application, relating to the field of biotechnology. The kit includes recombinant monomeric alpha-synuclein, ammonium sulfate and thioflavine T. The incubation conditions are modified in this application, allowing the detection of pre-formed fibrils diluted to 10 picogram per milliliter for the first time, with 1 microliter of sample translating to a total of 10 fg of pre-formed fibrils detected, with a sensitivity of fg level in a single cycle.

### CROSS-REFERENCE TO RELATED APPLICATIONS

This application claims priority to Chinese Patent Application No. 202310648092.3, filed on Jun. 1, 2023, the contents of which are hereby incorporated by reference.

### INCORPORATION BY REFERENCE STATEMENT

This statement, made under Rules 77(b)(5)(ii) and any other applicable rule incorporates into the present specification of an XML file for a “Sequence Listing XML” (see Rule 831(a)), submitted via the USPTO patent electronic filing system or on one or more read-only optical discs (see Rule 1.52(e)(8)), identifying the names of each file, the date of creation of each file, and the size of each file in bytes as follows:
File name: sequence_347-038_9957
Creation date: Aug. 8, 2023
Byte size: 2,186

### TECHNICAL FIELD

The present application relates to the field of biotechnology, and in particular to a kit for quiescent detecting alpha-synuclein aggregates.

### BACKGROUND

Parkinson's disease (PD) and related diseases such as Multiple System Atrophy (MSA) and Dementia with Lewy Bodies (DLB) are collectively known as alpha-synucleinopathy (α-synucleinopathy). This group of diseases is difficult to be diagnosed on the basis of clinical manifestations, physical symptoms, and routine examinations, while pathological α-synuclein (α-Syn) aggregates are the core markers of this group of diseases, and are also the golden standard in diagonalizing.

Nonetheless, conventional pathological α-syn detection, which is based on the principle of antigen-antibody binding, generally employs antibody-based techniques like enzyme-linked immuno sorbent assay (ELISA) and immunohistochemistry (IHC). It is difficult to use this technique, which only allows detection of nanogram (ng) level of pathological α-Syn in extracranial tissue of patients. Importantly, the α-Syn monomers and pathological α-Syn aggregates are capable of converting into each other and sharing the same antigenic epitopes, resulting in poor specificity of the antibody detection.

Well-established seed amplification assays (SAA), like protein misfolding cyclic amplification (PMCA) (first generation technology) and real-time quaking-induced conversion (RT-QuiC) assay (second generation technology) have successively become the latest α-Syn protein ultramicroscopic assays in recent years. Based on the conversion behaviour of α-syn, described as “prion-like”, meaning the α-Syn monomers are capable of converting into pathological α-Syn aggregates, has been reported by evidences of graft to host spreading of α-syn aggregates. The emergence of in vitro prion amplification detection methods based on prion-like seeding characteristics of α-Syn are utilized to carry out multiple amplification cycles to exponentially amplify the misfolded α-Syn, so as to efficiently amplify trace amounts of pathological α-Syn. These techniques employ recombinant α-Syn and sonicating or intermittent shaking to initiate the template-seeded aggregation of recombinant monomeric α-Syn into amyloid fibrils upon seeding by traces of α-Syn aggregates present in biospecimens. The newly generated β-sheet rich amyloid fibrils bind to an amyloid-sensitive dye, thioflavine T (ThT, amyloidogenic fibril-specific dye), resulting an enhanced fluorescence. The results of amplification are manifested by α-Syn aggregation kinetics, threshold of the kinetics and strong fluorescence signals from the final amplification products are employed for judging a positive result. This method has been successful in detecting misfolded prion protein in a variety of tissues and bodily fluids in central and peripheral tissues and body fluids in clinically diagnosed synucleinopathy cases at femtogram (fg) level, which is one million times that of the conventional antibody detection technique, making it a state-of-the-art method for the detection of α-Syn spectrum disorders.

The PMCA and RT-QuiC assay has recently emerged as a powerful platform for amplified detection of α-Syn aggregates; however, the thermal condition for α-Syn amplification in vitro is usually set at 37 degrees Celsius (° C.) or 42° C., a setting that makes it difficult to inactivate the biological activity of impurity proteins. Nevertheless, the detection process takes about 3-7 days, which seriously affects the efficiency and reproducibility of the amplification; it is therefore currently not applicable in clinical practice for uncapable of providing timely and accurate diagnosis value. More importantly, the PMCA and RT-QuiC assays rely on sonication or cyclic shaking to expose the specific epitopes of α-Syn, thus keeping the entire in vitro amplification reaction system in the extended maintenance phase. In addition, sonication or shaking disrupts the morphology of the tissue samples and prevents localization of the origination of the pathological α-Syn to the target tissue, thus limiting the clinical utilization.

### SUMMARY

The present application aims to provide a kit for quiescent detecting alpha-synuclein (α-Syn) aggregates, so as to solve the problems existing in the prior art; the kit for quiescent detecting α-Syn aggregates provided by the present application features high specificity and sensitivity, as well as rapidity and simplicity, making it an alternative of brain micro-biopsy (invasive) for detecting α-Syn aggregates in trace amounts of body fluids or micro-tissues.

To achieve the above objectives, the present application provides the following technical schemes:
the present application provides a kit for quiescent detecting α-Syn aggregates, where the kit includes recombinant monomeric α-Syn, ammonium sulfate (AS) and thioflavine T (ThT).

Optionally, the kit also includes Tris buffer solution.

Optionally, the kit also includes pre-formed fibrils (PFFs), and the PFFs are aggregates formed by self-aggregation of the recombinant monomeric α-Syn.

Optionally, a preparation method of the PFFs includes following steps:
(1) dissolving the recombinant monomeric α-Syn in the Tris buffer solution for consecutive shaking to form fibrils; and
(2) carrying out ultrasonic crushing on the fibrils to obtain the PFFs.

Optionally, in the step (1), a shaking environment is 37 degrees Celsius (° C.) with a duration of 7 days.

Optionally, a detection step of the kit includes:
S1, subjecting a sample to be detected to an incubation reaction in an incubation reaction solution; and
S2, detecting fluorescent signals after the incubation reaction is completed, and determining a positive result if a thioflavine T fluorescent signal amplification product is observed;
the incubation reaction solution includes 1 milligram per milliliter (mg/mL) recombinant monomeric α-Syn, 10 millimolar (mM) Tris-HCl with pH 7.5, 50 mM NaCl, 40 micromolar (μM) thioflavine T and 10 weight percentage (wt %) ammonium sulfate.

Optionally, in the step S1, a temperature of the incubation reaction is 70° C. and a duration is 3-12 hours (h).

The present application discloses the following technical effects:
by analyzing the physicochemical properties of α-Syn proteins, the present application improves the PMCA and RT-QuiC ultramicrographic examination protocols, firstly by changing the incubation solution system of the target proteins, and then successfully develops a brand new α-Syn amplification and detection technology, named as quiescent seeding amplification assay (QSAA); it is a third-generation technology for in vitro amplification of misfolded proteins, allowing the successful change of the bead-stirring incubation mode of pathological α-Syn aggregate to the quiescent incubation mode without beads. By adding ammonium sulphate and increasing the incubation temperature to the physiological protein inactivation temperature of 70° C., the amplification rate of pathological fibrils is greatly improved, and in situ incubation in intact tissue is achieved to accurately locate the site of lesions and to improve the specificity of the α-Syn assay, thereby overcoming the instability and inaccuracy of the original assay technology and broadening the scope of application.

PMCA, RT-QuiC detection technology requires cyclic crushing of tissue by shaking or sonication, so that the protein aggregation reaction is maintaining in the stage of fibril extension, which induces the continuous amplification of α-Syn. However, the detection method requires high-throughput ultrasound incubation equipment or disposable stirrer beads in conjunction with a specific shaking and stirring equipment, causing the detection operation to be very complicated and poorly reproducible. To this end, the quiescent incubation mode of the present application relies no longer on ultrasonic crushing or cyclic shaking for biological sample processing. therefore maintaining the integrity of the samples, and realizing the in situ amplification of α-Syn pathogenic proteins in biological samples of the brain and skin from patients with Parkinson's Disease (PD) and other related synucleinopathies, i.e., realizing the precise localization of the misfolded protein and pathologically-assisted diagnosis.

By changing the incubation conditions, the present application allows the detection of α-Syn PFFs diluted to 10 picogram per milliliter (pg/mL), and the total amount of PFFs detected is 10 fg PFFs converted from 1 microliter (μL) sample, with the sensitivity of a single cycle reaching the fg level. While the sensitivity of enzyme-linked immuno sorbent assay (ELISA) is 100 pg/mL-10 nanogram per milliliter (ng/mL), the converted total amount is 10 pg-1 ng based on 100 μL per well, indicating that the pathological α-Syn detection technology of the present application is significantly superior to ELISA, and suggesting that the optimized α-Syn ultramicroscopic technology features strong specificity and high sensitivity, as well as rapidity and simplicity, which can be used as an alternative for brain micro-biopsy (invasive) to detect the α-Syn aggregation in extracranial tiny tissues.

### BRIEF DESCRIPTION OF THE DRAWINGS

For a clearer description of the technical schemes in the embodiments or prior art of the present application, the accompanying drawings to be used in the embodiments are briefly described hereinafter, and it is obvious that the accompanying drawings in the description hereinafter are only some of the embodiments of the present application, and that for a person of ordinary skill in the field, other accompanying drawings are available on the basis of the accompanying drawings without any creative labour.

FIG. 1 is a schematic diagram illustrating a technical idea of the present application.

FIG. 2 shows a detection process of the present application.

### DETAILED DESCRIPTION OF THE EMBODIMENTS

Various exemplary embodiments of the present application are now described in detail, and this detailed description should not be considered as a limitation of the present application, but should be understood as a further detailed description of certain aspects, features, and embodiments of the present application.

It is to be understood that the terms described in the present application are only intended to describe particular embodiments and are not intended to limit the present application. Each smaller range between any stated value or intermediate value within the stated range, and any other stated value or intermediate value within the stated range, is also included within the present application. The upper and lower limits of these smaller ranges may be independently included or excluded from the range.

Unless otherwise indicated, all technical and scientific terms used herein have the same meaning as is commonly understood by those of ordinary skill in the art described in the present application. Although only preferred methods and materials are described herein, any methods and materials similar or equivalent to those described herein may also be used in the implementation or testing of the present application. All literature referred to in this specification is incorporated by reference for the purpose of disclosing and describing the methods and/or materials associated with the literature. In the event of conflict with any incorporated literature, the contents of this specification shall prevail.

Various improvements and variations are possible to the specific embodiments of the specification of the present application without departing from the scope or spirit of the present application, as will be apparent to a person skilled in the art. Other embodiments obtained from the specification of the present application are obvious to those skilled in the art. The specification and embodiments of the present application are exemplary only.

The terms “comprising”. “including”, “having” and “containing” used in this specification are all open terms, which means including but not limited to.

The experimental equipment used in the following embodiments includes, but is not limited to, biosafety cabinet, large shaking chamber, small shaking chamber, bacterial incubator, contact ultrasonic breaker, ultrahigh-speed centrifuge, high-speed centrifuge, table-top centrifuge, autoclave, water bath, stirrer, pH meter, scale, suction filtration pump, protein purifier, spectrophotometer, EP tube shaker, 4 degrees Celsius (° C.) refrigerator, −20° C. refrigerator, −80° C. refrigerator, PCR apparatus, tissue grinder, ice maker, electrophoresis apparatus, WB gel developer, non-contact ultrasonography, inverted fluorescence microscope, fluorescence enzyme marker and real-time fluorescence quantitative PCR apparatus. Availability and calibration of the experimental equipment are required in advance of testing.

Laboratory supplies include, but are not limited to, TB bacterial medium, sodium chloride, diluted hydrochloric acid, ammonium sulfate, SDS, NAOH, Ethylene Diamine Tetraacetic Acid (EDTA), protease inhibitors, fluorescent dye thioflavine T (ThT), Tris Buffer of pH 7.5, anionic columns, dry ice, disposable ring-drill skimmers, razor blades, and abrasive zirconia, and the laboratory supplies above need to be ready in advance and stored under appropriate conditions prior to testing.

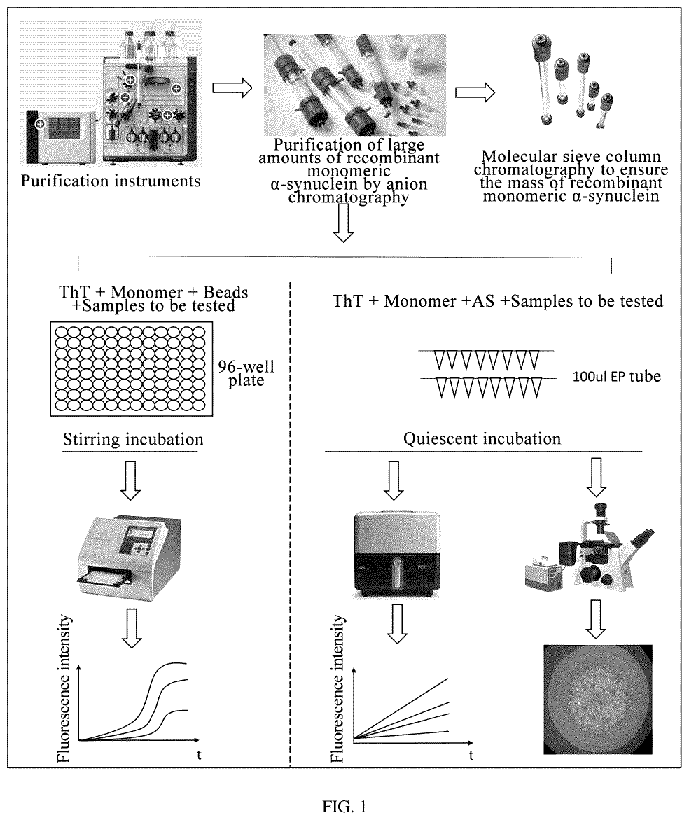

Principle of the present application: based on the replicative properties of α-Syn prion-like proteins, recombinant monomeric α-Syn can self-aggregate owing to the hydrophobic bonding of their NAC peptide fragments. In a proper environment of protein incubation solution, pathological α-Syn is self-assembled rapidly in vitro, i.e., recombinant monomeric α-Syn add tails at the ends of the aggregated fibrils to form rapid extensions. The aggregation property of α-Syn is optimized by changing the solution environment for conditional optimization, which enables rapid α-Syn amplification reactions, i.e., optimization of the external environment for α-Syn protein aggregation, which is greatly accelerated under conditions such as enhanced incubation temperature and addition of ammonium sulphate (AS). In the step of detection, α-Syn protein aggregates are capable of being assessed by fluorescence microscopy and the fluorescence signal of thioflavine T. As a fluorescent dye, the ThT binds to the β-sheet structure of amyloid fibrils and will instantly emit a fluorescent signal when it binds to α-Syn protein aggregates. The technical idea of the present application is schematically illustrated in FIG. 1.

Equipment and reagents required for this assay: fluorescence microscope, thermal incubator, α-Syn protein substrate (recombinant monomeric α-Syn), α-Syn pre-formed fibrils (PFFs), ammonium sulphate (AS), thioflavine T (ThT).
1. Fluorescence microscope: observing and recording the ThT signals and monitoring α-Syn aggregation process. In the process of the quiescent seeding amplification assay (QSAA), a fluorescence microscope is the main equipment for direct observation of the amplification products, e.g., recording fluorescence by means of the fluorescein isothiocyanate (FITC) fluorescence channel.
2. Thermal incubator: used to provide the specific temperature conditions required for the experiment. For QSAA, specific temperatures (e.g. 70° C.) are found to be the most suitable temperature for fibril extension.
3. α-Syn substrate: key reagent for QSAA detection, used for pathological α-Syn recruitment to form fibrils.
4. PFFs: in an aggregated form of α-Syn protein; as seeds, α-Syn protein self-replication is stimulated, which is used here as a positive control.
5. AS: it is found that AS significantly accelerates α-Syn fibril elongation.
6. ThT: a fluorescent probe that binds to the β-sheet structure of amyloid fibrils and visualizes the fibrils by fluorescence microscopy.

The above-mentioned equipment and reagents work together in QSAA detection, accelerating the aggregation and extension reaction of α-Syn protein quiescently by optimizing the external environment, and then detecting and recording the detection results through the observation of fluorescence microscope and ThT fluorescence signal.

### Embodiment 1 Preparation of Incubation Substrate (Recombinant Monomeric α-Syn)

Step 1: firstly, α-Syn recombinant prokaryotic expression plasmid is constructed, including amplifying α-Syn gene, with coding sequences (CDS) as shown in SEQ ID NO 1, by PCR and inserting it into the commercial pET28a vector by EcoRI and HindIII restriction enzyme sites to obtain the recombinant plasmid;

SEQ ID NO. 1:
atggatgtgttcatgaaaggactttcaaaggccaaggagggagttg
tggctgctgctgagaaaaccaagcagggtgtggcagaggcagctg
gaaagacaaaagagggagtcctctatgtaggttccaaaactaagg
aaggagtggttcatggagtgacaacagtggctgagacgaccaaag
agcaagtgacaaatgttggaggagcagtggtgactggtgtgacag
cagtcgctcagaagacagtggagggagctgggaatatagctgctg
ccactggctttgtcaagaaggaccagatgggcaagggtgaggagg
ggtacccacaggaaggaatcctggaagacatgcctgtggatcctg
gcagtgaggcttatgaaatgccttcagaggaaggctaccaagact
atgagcctgaagccta;

step 2:1 millimolar (mM) isopropyl β-D-1-Thiogalactopyranoside (IPTG) is used for prokaryotic induction expression of the recombinant plasmid obtained in the step 1 for 3 hours (h);
step 3:1 liter (L) of the bacterial solution is subjected to centrifugation at 6,000 g for 10 minutes (min), followed by resuspension of 50 milliliters (mL) of resuspension solution (10 mM Tris pH 7.5, 750 mM NaCl, 1×EDTA, 1× protease inhibitor);
step 4: a crushing process is carried out using ultrasound of 60% intensity (maximum intensity of the ultrasonic crusher of 1,000 Watts (W)) for a duration of 1 h;
step 5: the treated bacterial solution is boiled for 10 min, and then centrifuged and the supernatant is taken;
step 6: 50% AS is used for precipitation to obtain the target protein, followed by centrifugation and the precipitate is taken;
step 7: the pH is adjusted to 3 with diluted hydrochloric acid, the supernatant is taken after centrifugation, and then the pH is adjusted to 7.5 with sodium hydroxide;
step 8: the target proteins are obtained by eluting with 300 mM NaCl after passing through an anion-exchange column of 50 mM NaCl;
step 9: the target proteins are passed through a 50 kd ultrafiltration tube to obtain recombinant monomeric α-Syn;
step 10: the above recombinant monomeric α-Syn are mixed with Tris buffer solution with concentration adjusted to 4 mg/mL α-Syn, 10 mM Tris-HCl pH 7.5, and 100 mM NaCl;
step 11: the mixed solution obtained in step 10 is taken in 500 microliters (μL) in a 1.5 mL EP tube and shaken continuously at 1,000 revolutions per minute (rpm) for 7 days at 37° C. to form fibrils; and
step 12: the fibrils are crushed by ultrasound at 20% intensity (maximum intensity of the ultrasonic crusher of 1,000 W) for 2 min, operated as 1 second(s) on and 1 s off, to form positive control PFFs.

This method provides an efficient and reproducible approach to prepare recombinant monomeric α-Syn incubation substrates, providing a powerful tool for carrying out aggregation of α-Syn proteins.

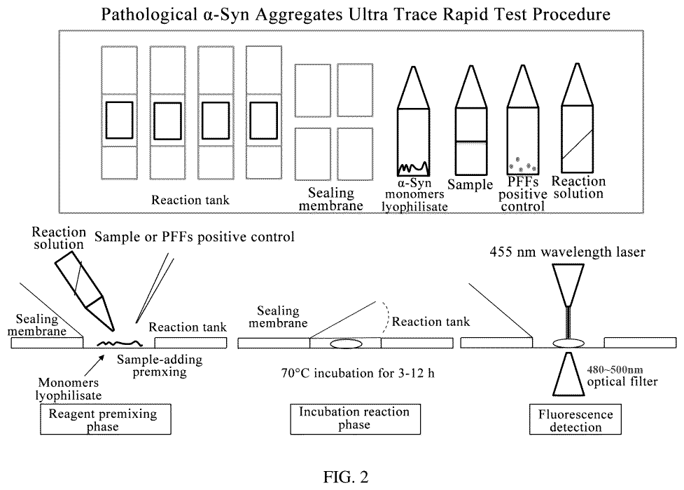

### Embodiment 2 Quiescent Amplification of Pathological α-Syn Aggregates in Skin Homogenates (See FIG. 2 for the Detection Process)

(1) Preparation of Skin Homogenate Samples of Patients with Synucleinopathies
a1: Skin samples of 3 mm×3 mm×3 mm are taken from the epidermis, dermis and adipose layer;
a2: the epidermal portion of the skin samples is removed using a razor blade;
a3: the samples are rinsed with saline to remove all residual blood;
a4: the skin tissue with size of 3 mm×3 mm×2 mm is placed in a grinding tube, and subjected to grinding by adding grinding balls and 700 μL of grinding Buffer (10 mM Tris pH7.5, 50 mM NaCl, 1× protease inhibitor), where the grinding programme is set as 75 hertz (Hz) with 45 s of work and 15 s of rest for a total of 5 cycles, and after the grinding is completed, centrifugation is performed at 3,000 g for 5 min, and the supernatant is taken to obtain the skin homogenate samples from the patients with synucleinopathies.

### (2) Sample Detection

Samples to be detected: skin homogenate samples of patients with synucleinopathies prepared in step (1); positive control: 1 nanogram per milliliter (ng/mL) PFFs; negative control: skin homogenate of patients not having synucleinopathies, the preparation method is the same as step (1).

The samples to be tested are tested as follows:
b1: the sample to be tested is thermally inactivated in water bath (or metal bath) at 70° C. for 5 min;
b2: an incubation reaction solution containing 10 mM Tris-HCl (pH 7.5), 50 mM NaCl, 1 mg/mL recombinant monomeric α-Syn, 40 micromolar (μM) ThT and 10 wt % AS is prepared;
b3: the sample to be tested after heat inactivation is taken as 1 μL, and mixed in 10 μL of the incubation reaction solution, and added to an 8-strip tube;
b4: the incubation reaction conditions are set as: quiescent incubation at 70° C. for 12 h; and
b5: after the incubation, 2 μL of the incubation product is aspirated and observed directly under a fluorescence microscope to verify the results.

Results determination: a large number of ThT fluorescence signal amplification products are found in skin homogenate samples from patients with synucleinopathies and in positive controls. No amplification products of any ThT-positive signals are found in the negative control group.

This embodiment provides a potential tool for early diagnosis and monitoring of disease progression in Parkinson's disease (PD) and Multiple System Atrophy (MSA) by efficiently amplifying and detecting pathological α-Syn aggregates in skin samples.

### Embodiment 3 In Situ Amplification of Pathological α-Syn Aggregates in Skin and Brain Tissue Sections

Samples to be tested: frozen brain tissue and skin samples of patients with PD, MSA and Dementia with Lewy Bodies (DLB); positive control: standard brain tissue or skin samples of patients with synucleinopathies; negative control: brain tissue or skin samples from patients not having synucleinopathies.

### Detection:

c1: frozen brain tissue and skin samples of 3 mm×3 mm×3 mm from patients with PD, MSA, and DLB disorders are encapsulated in embedding solution of optimal cutting temperature (OCT) and sliced into slices with thickness of 14 micrometers (μm);
c2: the sliced samples are pasted on 15 mm×15 mm slides;
c3: slides are placed in 24-well plates and incubated in buffer containing 1 mg/mL recombinant monomeric α-Syn, 10 wt % AS, 40 μM ThT, 50 mM NaCl, and 10 mM Tris-HCl (pH 7.5) in a total volume of 300 μL per well using a sealing membrane;
c4: the incubation reaction conditions are arranged as: quiescent incubation at 70° C. for 3 h; and
c5: after the incubation, the fluorescence image is collected through FITC channel with fluorescence microscope.

With the results, it is determined that a large number of amplification products with ThT-positive fluorescent signals are observed in brain tissue and skin samples from patients with PD, MSA and DLB, as well as in the positive control group. No amplification products with ThT-positive signals are found in the negative control group.

All these steps indicate that the quiescent incubation protocol effectively amplifies pathological α-Syn in brain and skin samples from PD patients in situ, possessing the ability to detect the disease in the early diagnosis of the disease and the assessment of disease progression.

### Embodiment 4

### Sensitivity Experiment:

step 1: gradient dilution of PFFS: in a polypropylene PCR tube, the gradient dilution (1:10, 1:100, 1:1,000, 1:10,000, 1:100,000, 1:1,000,000, 1:10,000,000, 1:100,000,000) of 1 mg/mL PFFs is carried out with 100 mM NaCl;
step 2: preparation of incubation reaction solution, containing 0 mM Tris-HCl (pH 7.5), 100 mM NaCl, 1 mg/mL recombinant monomeric α-Syn, 40 uM ThT and 10 wt % AS;
step 3: separately, 1 μL of the gradient-diluted PFFs is mixed in 10 μL of incubation reaction solution and added to an 8-strip tube;
step 4: incubation reaction conditions: quiescent incubation at 70° C. for 12 h;
step 5: after incubation, 2 μL of the incubation products are aspirated separately and observed directly under a fluorescence microscope to verify the results.

Results determination: the ThT fluorescence signal amplification product exhibits filamentous fluorescence that decreases with gradient dilution until no filaments are visible under the microscope. The lowest concentration at which fluorescence is observed is 10 μg/mL, and 1 μL of sample translates to a total of 10 fg of PFFs detected, with a sensitivity of fg level for a single cycle.

The above-mentioned embodiments only describe the preferred mode of the present application, and do not limit the scope of the present application. Under the premise of not departing from the design spirit of the present application, various modifications and improvements made by ordinary technicians in the field to the technical scheme of the present application shall fall within the protection scope determined by the claims of the present application.

What is claimed is:

1. A kit for quiescent detecting alpha-synuclein aggregates, wherein the kit comprises recombinant monomeric alpha-synuclein, ammonium sulfate and thioflavine T.

2. The kit for quiescent detecting alpha-synuclein aggregates according to claim 1, wherein the kit also comprises Tris buffer solution.

3. The kit for quiescent detecting alpha-synuclein aggregates according to claim 1, wherein kit also comprises pre-formed fibrils, and the pre-formed fibrils are aggregates formed by self-aggregation of the alpha-synuclein monomers.

4. The kit for quiescent detecting alpha-synuclein aggregates according to claim 3, wherein a preparation method of the pre-formed fibrils comprises following steps:
(1) dissolving the recombinant monomeric alpha-synuclein in Tris buffer solution for shaking culture to form fibrils; and
(2) carrying out ultrasonic crushing on the fibrils to obtain the pre-formed fibrils.

5. The kit for quiescent detecting alpha-synuclein aggregates according to claim 4, wherein in the step (1), a temperature of the shaking culture is 37 degrees Celsius and a duration is 7 days.

6. The kit for quiescent detecting alpha-synuclein aggregates according to claim 1, wherein detecting steps of the kit comprise:
S1, subjecting a sample to be detected to an incubation reaction in an incubation reaction solution; and
S2, detecting fluorescent signals after the incubation reaction is completed, and determining a positive result if a thioflavine T fluorescent signal amplification product is observed;
wherein the incubation reaction solution comprises 1 milligram per milliliter recombinant monomeric alpha-synuclein, 10 millimolar Tris-HCl with pH 7.5, 50 millimolar NaCl, 40 micromolar thioflavine T and 10 weight percentage ammonium sulfate.

7. The kit for quiescent detecting alpha-synuclein aggregates according to claim 6, wherein in the step S1, a temperature of the incubation reaction is 70 degrees Celsius and a duration is 3-12 hours.
